SEQUENCE LISTING (13 sequences)

SEQ ID NO: 1 - DNA, 1398 bp, Unknown
  Bifidobacterium longum
catgcagtcgaacgggatccatcaagcttgcttggtggtgagagtggcgaacgggtgagtaatgcgtgaccgacctgccccatacaccggaatagctcctggaaacgggtggtaatgccggatgttccagttgatcgcatggtcttctgggaaagctttcgcggtatgggatggggtcgcgtcctatcagcttgacggcggggtaacggcccaccgtggcttcgacgggtagccggcctgagagggcgaccggccacattgggactgagatacggcccagactcctacgggaggcagcagtggggaatattgcacaatgggcgcaagcctgatgcagcgacgccgcgtgagggatggaggccttcgggttgtaaacctcttttatcggggagcaagcgtgagtgagtttacccgttgaataagcaccggctaactacgtgccagcagccgcggtaatacgtagggtgcaagcgttatccggaattattgggcgtaaagggctcgtaggcggttcgtcgcgtccggtgtgaaagtccatcgcttaacggtggatccgcgccgggtacgggcgggcttgagtgcggtaggggagactggaattcccggtgtaacggtggaatgtgtagatatcgggaagaacaccaatggcgaaggcaggtctctgggccgttactgacgctgaggagcgaaagcgtggggagcgaacaggattagataccctggtagtccacgccgtaaacggtggatgctggatgtggggcccgttccacgggttccgtgtcggagctaacgcgttaagcatcccgcctggggagtacggccgcaaggctaaaactcaaagaaattgacgggggcccgcacaagcggcggagcatgcggattaattcgatgcaacgcgaagaaccttacctgggcttgacatgttcccgacgatcccagagatggggtttcccttcggggcgggttcacaggtggtgcatggtcgtcgtcagctcgtgtcgtgagatgttgggttaagtcccgcaacgagcgcaaccctcgccccgtgttgccagcggattgtgccgggaactcacgggggaccgccggggttaactcggaggaaggtggggatgacgtcagatcatcatgccccttacgtccagggcttcacgcatgctacaatggccggtacaacgggatgcgacgcggcgacgcggagcggatccctgaaaaccggtctcagttcggatcgcagtctgcaactcgactgcgtgaaggcggagtcgctagtaatcgcgaatcagcaacgtcgcggtgaatgcgttcccgggccttgtacacaccgcccgtcaagtcatgaaagtgggcagcacccgaagccggtggcctaaccccttgtgggatggagccgtctaa

SEQ ID NO: 2 - DNA, 20 bp, Unknown
  27F
agagtttgatcctggctcag

SEQ ID NO: 3 - DNA, 19 bp, Unknown
  518F
ccagcagccgcggtaatac

SEQ ID NO: 4 - DNA, 20 bp, Unknown
  907R
ccgtcaattcmtttragttt

SEQ ID NO: 5 - DNA, 19 bp, Unknown
  1492R
ggttaccttgttacgactt

SEQ ID NO: 6 - DNA, 21 bp, Unknown
  Primer for beta-actin (sense)
tctacgaggggtatgccctcc

SEQ ID NO: 7 - DNA, 21 bp, Unknown
  Primer for beta-actin (anti-sense)
ggatgccacaggactccatgc

SEQ ID NO: 8 - DNA, 21 bp, Unknown
  Primer for ocln (sense)
gacttcaggcagcctcgttac

SEQ ID NO: 9 - DNA, 22 bp, Unknown
  Primer for ocln (anti-sense)
gccagttgtgtagtctgtctca

SEQ ID NO: 10 - DNA, 19 bp, Unknown
  Primer for cldn4 (sense)
tggggctacaggtaatggg

SEQ ID NO: 11 - DNA, 21 bp, Unknown
  Primer for cldn4 (anti-sense)
ggtctgcgaggtgacaatgtt

SEQ ID NO: 12 - DNA, 22 bp, Unknown
  Primer for has2 (sense)
ctcttttggactgtatggtgcc

SEQ ID NO: 13 - DNA, 22 bp, Unknown
  Primer for has2 (anti-sense)
agggtaggttagccttttcaca

(19) United States
(12) Patent Application Publication
(10) Pub. No.: US 2022/0143112 A1
(43) Pub. Date: May 12, 2022

(54) NOVEL BIFIDOBACTERIUM LONGUM STRAIN OR COSMETIC COMPOSITION COMPRISING SAME

(71) Applicant: ATOGEN CO., LTD, Daejeon (KR)

(72) Inventors: Yong Hyun LEE, Daejeon (KR); Bo Ram BECK, Daejeon (KR); Ji Hee KANG, Daejeon (KR); Do Yeun JEONG, Daejeon (KR); Gun Seok PARK, Daejeon (KR); Sung Hoon IM, Daejeon (KR); Won Ho SONG, Daejeon (KR)

(73) Assignee: ATOGEN CO., LTD, Daejeon (KR)

(21) Appl. No.: 17/600,650

(22) PCT Filed: Jun. 10, 2019
(86) PCT No.: PCT/KR2019/006938

§ 371 Date: Oct. 1, 2021

(30) Foreign Application Priority Data
Apr. 2, 2019 (KR) 10-2019-0038337

(51) Int. Cl. A61K 35/745; A23L 33/135; A61P 17/10; A61P 17/18; A61P 29/00; C12N 1/20

(52) U.S. Cl. CPC A61K 35/745; A23L 33/135; A61P 17/10; C12R 2001/01; A61P 29/00; C12N 1/205; A23Y 2300/55; A61P 17/18

(57) ABSTRACT

The present invention relates to a novel Bifidobacterium longum ATG-F5 strain. The F5 strain is a functional beneficial bacterium which is safe from antibiotic resistance, improves skin health through antibacterial activity against the skin pathogen Cutibacterium acnes, an antioxidant effect on radicals, skin barrier improvement, and anti-inflammatory effects, and enhances skin beauty through a skin whitening function, a wrinkle improvement function, and a dry skin alleviation function. The strain may be used in a cosmetic composition or health functional food.

### TECHNICAL FIELD

The present invention relates to a novel Bifidobacterium longum ATG-F5 (Bifidobacterium longum ATG-F5, accession no. KCTC13828BP) strain or a cosmetic composition containing the same.

### BACKGROUND ART

In the field of skin care, various care techniques are used as the scope of application thereof is gradually expanded. Among the various techniques, people prefer cosmetics with whitening functional materials that brighten the skin or cosmetics with function of wrinkle improvement and suppression that create youthful-looking skin and promote skin elasticity. Recently, functional cosmetics that function as skin barrier and prevent dry skin have also appeared, and whitening and wrinkle-improving cosmetics are being developed with more various functionalities due to shifts in people's perception.

Among these techniques, skin-whitening cosmetics are based on the principle of melanin activity inhibition that determines the color of human skin. Melanin is a pigment that is found in the basal layer of the epidermis and blocks the sun's ultraviolet (UV) rays. Yet, when physiological functions of the skin deteriorated due to excessive synthesis or aging, melanin settles into the skin, causing spots, freckles, and various pigments.

Melanin synthesis is modulated by tyrosinase, tyrosinase related protein-1 (TRP-1), tyrosinase related protein-2 (TRP-2), adrenocorticotropic hormone (ACTH) which is an inducer of cyclic adenosine monophosphate (cAMP), and α-melanocyte stimulating hormone (α-MSH) (Chan et al., 2011). Especially, tyrosinase, tyrosinase related protein-1 (TRP-1), and tyrosinase related protein-2 (TRP-2) modulate the stimulation of melanin production. By promoting hydroxylation reaction, tyrosinase catalyzes the conversion of tyrosine to 3,4-dihydroxyphenylalanine (DOPA) and DOPA to DOPA-quinone. (Jang and Park, 2017) This increased DOPA-quinone leads to melanin production. Thus, the inhibition of tyrosinase activity plays an important role in skin whitening by inhibiting melanin production.

Cosmetics with the function of wrinkle improvement and suppression are based on the principle involving collagen, well-known for skin elasticity. There are two types of skin aging: intrinsic and extrinsic. Intrinsic aging occurs naturally as we grow older, whereas extrinsic aging or photoaging is caused by exposure to ultraviolet (UV) light. Human skin is composed of three layers of tissue: the epidermis, the dermis, and subcutaneous fat. The epidermis consists mostly of keratinocytes to protect skin and the dermis is associated with skin elasticity and wrinkles. The dermis manifests fibrous proteins collagen and elastin. Collagen is a fibrous protein, and there are 14 types, of which type 1 collagen acting on the skin is mainly present. (Kim and Yoon., 2013) Matrix metalloproteinases (MMPs) are matrix proteins that degrade collagen, and more than 20 types of MMPs are known. When MMP-1, also known as collagenase-1, degrades collagen, skin elasticity decreases and wrinkles are formed. (Lee et al., 2013) Therefore, it is possible to reduce wrinkles and maintain skin elasticity by regulating the proteins that act on the skin.

In addition to the above functions related to skin care, there are newly emerging functions such as skin barrier strengthening and dry skin alleviation. Improving skin barrier function involves improving the skin condition while enhancing the expression of occludin and claudin, proteins required when tight junction protein is involved in cell-to-cell contact in epithelial cells, which are skin tissues. (B. Tebbe et al., 2002) Dry skin alleviation function involves inducing hyaluronan synthase 2 (HAS2) hyaluronic acid (HA) synthesis and thus reducing moisture loss in the skin. HA carries out an important biological role in the skin such as retaining moisture, maintaining tissue structure and elasticity, and exchanging nutrients.

Accordingly, the present invention proposes Bifidobacterium longum ATG-F5 strain deposited with an accession number of KCTC13828BP as a way to enhance functions related to skin beauty including skin whitening, wrinkle improvement, skin barrier strengthening, dry skin alleviation, anti-inflammatory, and anti-acne functions.

### RELATED ART LITERATURE

### Patent Literature

(Patent Literature 1) Republic of Korea Patent Registration No. 10-1589464 (Name of the invention: A novel bifidus strain and a functional food composition for promoting growth including the same, Applicant: Cell Biotech Co., Ltd., registration date: Jan. 22, 2016)
(Patent Literature 2) European Patent Registration No. 2308566B1 (Name of invention: Use of orally administered probiotic bifidobacteria for human beauty benefits, Applicant: The Procter & Gamble Company and others, registration date: Apr. 13, 2011)
(Patent Literature 3) Korean Registered Patent No. 10-0889605 (Name of invention: Cosmetic composition for wrinkle improvement, Applicant: LG Household & Health Care Co., Ltd., registration date: Mar. 12, 2009)
(Patent Literature 4) Korean Registered Patent No. 10-1456040 (Name of invention: Cosmetic composition for whitening, Applicant: LG Household & Health Care Co., Ltd., registration date: Oct. 23, 2014)
(Patent Literature 5) Republic of Korea Patent Publication No. 10-2016-0092564 (Name of the invention: A cosmetic composition having antioxidant, wrinkle improvement and moisturizing effect containing a two-stage fermentation lysate of yeast and lactic acid bacteria, Applicant: Incos, Release date: Aug. 5, 2016)
(Patent Literature 6) Korean Patent Registration No. 10-1788544 (Name of the invention: Cosmetic composition for strengthening skin barrier containing complex lactic acid bacteria culture, Applicant: Cosmax Co., Ltd. and one other, registration date: Oct. 16, 2017)
(Patent Literature 7) Korean Registered Patent No. 10-1885195 (Name of invention: Cosmetic composition containing Mokseo fermented extract as an active ingredient, Applicant: Koreana Cosmetics Co., Ltd., registration date: Jul. 30, 2018)

### Non-Patent Literature

(Non-Patent Literature 1) B. Tebbe J. Mankertz C. Schwarz S. Amasheh M. Fromm C. Assaf U. Schultz-Ehrenburg H. Sanchez Ruderisch J.-D. Schulzke C. Orfanos. (2002). Tight junction proteins: a novel class of integral membrane proteins. Archives of Dermatological Research, 294(1-2), 14-18.
(Non-Patent Literature 2) Chan, Y. Y., Kim, K. H., & Cheah, S. H. (2011). Inhibitory effects of Sargassum polycystum on tyrosinase activity and melanin formation in B16F10 murine melanoma cells. Journal of Ethnopharmacology, 137(3), 1183-1188.
(Non-Patent Literature 3) Chang, O. K., Seol, K. H., Jeong, S. G., Oh, M. H., Park, B. Y., Perrin, C., & Ham, J. S. (2013). Casein hydrolysis by Bifidobacterium longum KACC91563 and antioxidant activities of peptides derived therefrom. Journal of dairy science, 96(9), 5544-5555.
(Non-Patent Literature 4) Choi, S. M., Jang, H. S., & Kim, B. O. (2008). Effects of H2O2 and chlorhexidine on MMP-1, TIMP-1, 2, Type 1 collagen, fibronectin and UNCL expressions in human periodontal ligament fibroblasts. The Journal of the Korean Academy of Periodontology, 38(4), 645-656.
(Non-Patent Literature 5) EFSA. (2012). Guidance on the assessment of bacterial susceptibility to antimicrobials of human and veterinary importance. EFSA Journal 10(6):2740.
(Non-Patent Literature 6) Erel, O. (2004). A novel automated direct measurement method for total antioxidant capacity using a new generation, more stable ABTS radical cation. Clinical biochemistry, 37(4), 277-285.
(Non-Patent Literature 7) Hasegawa, T. (2010). Tyrosinase-expressing neuronal cell line as in vitro model of Parkinson's disease. International journal of molecular sciences, 11(3), 1082-1089.
(Non-Patent Literature 8) Gye Won Lee, Sung Min Park, Yung Choon Yoo, and Young Ho Cho. (2013). Effect of Ponciri Fructus Extracts Fermented with Ganoderma lucidum on the Collagen Synthesis and Expression of Matrix Metalloproteinase-1. Kor Society for Biotechnology and Bioengineering Journal 28(2), 106-114.
(Non-Patent Literature 9) Kim, Y. J., & Yoon, Y. (2013). Regulation of Col1A1 and MMP1 Expression by Taurine, Major Component of Oyster, in Human Dermal Fibroblasts. Kor J Aesthet Cosmetol, 11(2), 393-397.
(Non-Patent Literature 10) Sang-Myeoung Nam, Il-Jun Kang and Mee-Hye Shin. (2015). Anti-diabetic and Anti-oxidative Activities of Extracts from Crataegus pinnatifida. J East Asian Soc Dietary Life 25(2): 270-277.
(Non-Patent Literature 11) Re, R., Pellegrini, N., Proteggente, A., Pannala, A., Yang, M., & Rice-Evans, C. (1999). Antioxidant activity applying an improved ABTS radical cation decolorization assay. Free radical biology and medicine, 26(9-10), 1231-1237.
(Non-Patent Literature 12) Tae Won Jang and Jae Ho Park. (2017). Antioxidative Activities and Whitening Effects of Ethyl Acetate Fractions from The Immature Seeds of Abeliophyllum distichum. Journal of Life Science, 27(5), 536-544.

### DISCLOSURE

### Technical Problem

The objective of the present invention is to provide a novel Bifidobacterium longum ATG-F5 strain or a cosmetic composition containing the same.

### Technical Solution

The present invention relates to a novel Bifidobacterium longum ATG-F5 (Bifidobacterium longum ATG-F5, accession no. KCTC13828BP) strain.

The strain strengthens the skin barrier by boosting gene expression of occludin (OCLN) and claudin 4 (CLDN4) and alleviating skin dryness by encouraging gene expression of hyaluronan synthase 2 (HAS2).

The strain has an anti-acne function that inhibits the growth of the acne-causing bacterium Cutibacterium acnes, and has an anti-inflammatory activity that increases interleukin 10 (IL-10).

The strain also has a skin whitening function by inhibiting tyrosinase activity and controlling melanin production and a wrinkle improvement function by promoting pro-collagen through suppression of MMP-1 activity.

In one aspect of the present invention, the Bifidobacterium longum ATG-F5 strain may be replaced with or include at least one selected from the group consisting of: bacterial cells of the strain; a lysate of the cells; a culture of the strain; a culture solution in which cells are removed from the culture of the strain; a cell extract of the strain; an extract of the culture of the strain; and an extract of the culture solution from which the cells have been removed from the culture of the strain.

Accordingly, the present invention provides a cosmetic composition that contains the strain and has effects of skin barrier strengthening, dry skin alleviation, anti-inflammatory, anti-acne, antioxidant, and wrinkle improvement.

A formulation of the cosmetic composition may be selected from the group consisting of skin lotion, skin softener, skin toner, astringent, milk lotion, moisture lotion, nourishing lotion, massage cream, nourishing cream, moisture cream, hand cream, foot cream, neck cream, foundation, essence, pack, soap, cleansing foam, cleansing lotion, cleansing cream, body lotion, hair shampoo, hair treatment, hair conditioner, and body cleanser.

In another aspect of the present invention, there is provided health functional food containing the strain and having functions such as skin barrier strengthening, dry skin alleviation, anti-inflammatory, anti-acne, antioxidant, and wrinkle improvement.

The health functional food may be selected from the group consisting of meat, sausage, bread, candies, snacks, noodles, ice cream, dairy products, fermented milk, soups, ionized beverages, beverages, alcoholic beverages, gum, tea, and vitamin complexes.

The present invention also pertains to a composition containing the strain and for preventing or treating a disease selected from the group consisting of dry skin, acne and an inflammatory disease.

The inflammatory disease may be selected from the group consisting of dry skin, acne and an inflammatory disease, which is selected from the group consisting of inflammatory bowel disease, inflammatory collagen vascular disease, glomerulonephritis, inflammatory skin diseases, sarcoidosis, retinitis, gastritis, hepatitis, enteritis, arthritis, tonsillitis, pharyngitis, bronchitis, pneumonia, pancreatitis, sepsis, cystitis, nephritis and neuritis.

Hereinafter, the present invention will be described in more detail.

Bifidobacterium longum ATG-F5 strain of the present invention may be cultured in liquid or solid medium (broth or agar) of BL medium (hereinafter refer to as BL) or MRS medium (hereinafter refer to as MRS-cys) containing 0.3 to 0.7 g/L of L-cystein, and in BL or MRS-cys broth, Bifidobacterium longum ATG-F5 strain may be cultured to a concentration of 8.0×10⁸ to 1.0×10⁹ CFU/ml. When L-cystein is added to MRS medium, adding 0.6 g/L is preferable.

It is preferable that the strain be cultured at 30° C. to 37° C. for 16 to 24 hours, and the optimum temperature for culturing is 37° C., the minimum temperature is 15° C., the maximum temperature is 37° C., the optimum pH is 6.8, the minimum culturable pH is 5.0, and the maximum pH is 7.3. The optimal incubation time is 18 hours, the minimum incubation time is 8 hours, and the maximum incubation time is 72 hours.

The strain of the present invention is a safe strain that does not have resistance to antibiotics which at least one are selected from the group consisting of ampicillin, vancomycin, gentamicin, kanamycin, streptomycin, clindamycin, erythromycin, tetracycline, and chloramphenicol.

The present invention provides various compositions containing a Bifidobacterium longum ATG-F5 strain, that is, one or more selected from the group consisting of: bacterial cells of the strain; a lysate of the cells; a culture of the strain; a culture solution in which cells are removed from the culture of the strain; a cell extract of the strain; an extract of the culture of the strain; and an extract of the culture solution from which the cells have been removed from the culture of the strain.

Among them, the cell lysate of the F5 strain may be obtained after the cells are separated from the culture supernatant by centrifuging the culture solution of the strain, suspended in PBS, treated with Lysozyme, and sonicated. When the cells are separated by centrifuging, the cells may be washed with buffers such as PBS and centrifuged again. Centrifugation may be performed at 3,000 to 5000 rpm for 5 to 60 minutes, respectively. It is preferable that the lysozyme treatment of the cells suspended in PBS be performed at a concentration of 300 to 700 mg/ml at 30° C. to 37° C. and the ultrasonic treatment conditions are 50 to 90 W, 20 to 60 seconds, and 20 to 40 cycles. At this time, it is preferable to heat-treat for the death of microorganisms which may be alive in the cell lysate. It may be heat-treated at 50° C. to 70° C. for 30 to 60 minutes, and it is better to heat with a bath to minimize protein denaturation rather than direct heating. In addition, the separated culture supernatant may be used as a composition derived from various cultures by adjusting pH to 7 to 8 and filtering (using a 0.2 μm pore filter).

Each composition derived from the strain may be used without additional processing, purified or dried through various methods, and added to a cosmetic composition, a pharmaceutical composition or a health functional food.

Each composition derived from the strain may be dried by performing freeze drying, and may include excipients used when drying common strains or compositions derived from them. The excipients for freeze drying may be selected from gluconic acid, alginic acid, sodium alginate, hydroxypropyl cellulose, hydroxypropyl methyl cellulose, sodium carboxymethyl cellulose, methyl cellulose, carbomer, hyaluronic acid, tragacanth, karaya gum, water-soluble starch, pectin, gelatin, polyvinyl alcohol, polyvinyl pyrrolidone, oligosaccharides, sugar alcohols, calcium gluconate, calcium lactate, polymethyl methacrylate, wheat protein, soybean protein, methylcellulose, aquacoat, guar gum, locust bean gum, xanthan gum, gellan gum, gum arabic, trehalose, and the like.

The present invention may provide a cosmetic composition containing compositions derived from the strain, the cosmetic composition may be used as a composition for enhancing skin barrier, relieving skin dryness, anti-inflammatory, anti-acne, antioxidant, or wrinkle improvement effect. When the total weight of the cosmetic composition is based on 100% by weight, 0.001% to 20% by weight of various compositions derived from the strain may be included. The formulation of the cosmetic composition may be preferably selected from the group consisting of skin lotion, skin softener, skin toner, astringent, milk lotion, moisture lotion, nourishing lotion, massage cream, nourishing cream, moisture cream, hand cream, foot cream, neck cream, foundation, essence, pack, soap, cleansing foam, cleansing lotion, cleansing cream, body lotion, hair shampoo, hair treatment, hair conditioner, and body cleanser.

The cosmetic composition of the present invention may further include a component selected from the group consisting of water-soluble vitamins, oil-soluble vitamins, polymer peptides, polymer polysaccharides, sphingolipids, and seaweed extract.

The water-soluble vitamin may be any one that can be blended in cosmetics, but preferably vitamin B1, vitamin B2, vitamin B6, pyridoxine, pyridoxine hydrochloride, vitamin B12, pantothenic acid, nicotinic acid, nicotinic acid amide, folic acid, vitamin C, vitamin H, etc. In addition, the salts (thiamine hydrochloride, sodium ascorbate, etc.) thereof and derivatives (ascorbic acid-2-phosphate sodium salt, ascorbic acid-2-phosphate magnesium salt, etc.) are also included in the water-soluble vitamins used in the present invention. Water-soluble vitamins may be obtained by conventional methods such as a microbial transformation method, a purification from a culture of microorganisms, an enzyme method or a chemical synthesis method.

The oil-soluble vitamin may be any one that can be blended in the cosmetic composition, but preferably vitamin A, carotene, vitamin D2, vitamin D3, vitamin E (d1-alpha tocopherol, d-alpha tocopherol, d-alpha tocopherol), etc. and their derivatives (ascorbine palmitate, ascorbine stearate, ascorbine dipalmitate, d1-alpha tocopherol acetate, nicotinic acid d1-alpha tocopherol vitamin E, DL-pantotenyl alcohol, D-pantotenyl alcohol, pantotenyl ethyl ether, etc.) are also included in the oil-soluble vitamin used in the present invention. Oil-soluble vitamins can be obtained by conventional methods such as a microbial transformation method, a purification from a microorganism culture, an enzyme method or a chemical synthesis method.

The polymeric peptide may be any one that can be blended in the cosmetic composition, but preferably, collagen, hydrolyzed collagen, gelatin, elastin, hydrolyzed elastin, keratin, and the like are exemplified. The polymeric peptide may be purified and obtained by a conventional method such as a purification method from a culture medium of a microorganism, an enzyme method, or a chemical synthesis method, or it may be used after being purified from natural products such as dermis of pigs and cattle, and silk fibers of silkworms.

The polymer polysaccharide may be any one that can be blended in the cosmetic composition, but preferably, hydroxyethyl cellulose, xanthan gum, sodium hyaluronate, chondroitin sulfuric acid or a salt thereof (sodium salt, etc.) may be mentioned. For example, chondroitin sulfate or a salt thereof may be used after being purified from mammals or fish.

The sphingolipids may be any one that can be blended in the cosmetic composition, and ceramide, phytosphingosine, sphingoglycolipids, and the like are preferably mentioned. Sphingolipids are usually purified from mammals, fish, shellfish, yeast, plants, etc. by a conventional method, or may be obtained by chemical synthesis.

The seaweed extract may be any one that can be blended in the cosmetic composition, but preferably brown algae extract, red algae extract, green algae extract, etc. are exemplified. Carrageenan, alginic acid, sodium alginate, and potassium alginate, which are purified from these seaweed extracts are also included in the seaweed extract used in the present invention. Seaweed extract may be obtained by purifying from seaweed by a conventional method.

The cosmetic composition of the present invention may also contain other ingredients that are blended in a conventional cosmetic composition.

Other ingredients that may be added include fats and oils, moisturizers, emollients, surfactants, organic and inorganic pigments, organic powders, ultraviolet absorbers, preservatives, fungicides, antioxidants, plant extracts, pH adjusters, alcohols, pigments, fragrances, blood circulation promoters, cooling agents, antiperspirants, and purified water.

The oil and fat ingredients include an ester oil, a hydrocarbon oil, silicone grease, fluorine-based fats, animal fats, and plant fats.

The oil and fat ingredients include an ester oil, a hydrocarbon oil, silicone grease, fluorine-based fats, animal fats, and plant fats. Examples of the ester oil include glyceryl tri(2-ethylhexanoate), cetyl 2-ethylhexanoate, isopropyl myristate, butyl myristate, isopropyl palmitate, ethyl stearate, octyl palmitate, isocetyl isostearate, butyl stearate, ethyl linoleate, isopropyl linoleate, ethyl oleate, isocetyl myristate, isostearyl myristate, isostearyl palmitate, octyldodecyl myristate, isocetyl isostearate, diethyl sebacate, diisopropyl adipate, isoalkyl neopentanoate, tri(caprylic acid, capric acid) glyceryl, trimethylolpropane 2-ethylhexanoate, trimethylol propane triisostearate, pentaelysitol tetra (2-ethylhexanoate), cetyl caprylate, decyl laurate, hexyl laurate, myristic acid decyl, myristyl myristate, cetyl myristate, stearyl stearate, decyl oleate, cetyl ricinoleate, isostearyl laurate, isotridecyl myristate, isocetyl palmitate, octyl stearate, isocetyl stearate, isodecyl oleate, octyldodecyl oleate, octyldodecyl linoleate, isopropyl isostearate, cetostearyl 2-ethylhexanoate, stearyl 2-ethylhexanoate, hexyl isostearate, ethylene glycol dioctanoate, ethylene glycol dioleate, propylene glycol dicapric acid, propylene glycol di(caprylic, capric acid), propylene glycol dicaprylate, neopentyl glycol dicapric acid, neopentyl glycol dioctanoate, glyceryl tricaprylate, glyceryl triundecyl acid, glyceryl triisopalmitate, glyceryl triisostearate, octyldodecyl neopentanoate, isostearyl octanoate, octyl isononanoate, hexyldecyl neodecanoate, octyldodecyl neodecanoate, isocetyl isostearate, isostearyl isostearate, octyldecyl isostearate, polyglycerol oleic acid ester, polyglycerol isostearic acid ester, triisocetyl citrate, triisoalkyl citrate, triisooctyl citrate, lauryl lactate, myristyl lactate, cetyl lactate, octyldecyl lactate, triethyl citrate, acetyl triethyl citrate, acetyl tributyl citrate, trioctyl citrate, diisostearyl malate, 2-ethylhexyl hydroxystearate, di2-ethylhexyl succinate, diisobutyl adipate, diisopropyl sebacate, dioctyl sebacate, cholesteryl stearate, cholesteryl isostearate, cholesteryl hydroxystearate, cholesteryl oleate, dihydrocholesteryl oleate, phytsteryl isostearate, phytsteryl oleate, 12-stealoylhydroxystearate isocetyl, 12-stealoylhydroxystearate stearate, and 12-stealoylhydroxystearate isostearyl.

The hydrocarbon oil may include squalene, liquid paraffin, alpha-olefin oligomer, isoparaffin, ceresin, paraffin, liquid isoparaffin, polybudene, microcrystalline wax, and petrolatum.

The silicone grease may include polymethylsilicone, methylphenylsilicone, methylcyclopolysiloxane, octamethylpolysiloxane, decamethylpolysiloxane, dodecamethylcyclosiloxane, dimethylsiloxane-methylcetyloxysiloxane copolymer, dimethylsiloxane-methylstealloxysiloxane copolymer, alkyl-modified silicone oil, and amino-modified silicone oil.

The fluorine-based fats may include perfluoropolyether.

The animal and plant fats may include avocado oil, almond oil, olive oil, sesame oil, rice bran oil, bird flower oil, soybean oil, corn oil, rapeseed oil, apricot kernel oil, palm kernel oil, palm oil, castor oil, sunflower oil, grape seed oil, cottonseed oil, palm oil, kukui nut oil, wheat germ oil, rice germ oil, shea butter, evening primrose oil, macadamia nut oil, meadowfoam oil, egg yolk oil, tallow, horse oil, mink oil, orange raffia oil, jojoba oil, candelilla wax, carnauba wax, liquid lanolin, and hydrogenated castor oil.

The moisturizing agents may include a water-soluble low-molecular moisturizer, a fat-soluble molecular moisturizer, a water-soluble polymer, and a fat-soluble polymer.

The water-soluble low-molecular moisturizer may include serine, glutamine, sorbitol, mannitol, pyrrolidone sodium carboxylate, glycerin, propylene glycol, 1,3-butylene glycol, ethylene glycol, polyethylene glycol B (polymerization degree n=2 or more), polypropylene glycol (polymerization degree n=2 or more), polyglycerin B(polymerization degree n=2 or more), lactic acid, and lactate.

The fat-soluble molecular moisturizer may include cholesterol and cholesterol ester.

The water-soluble polymer may include carboxyvinyl polymer, polyaspartate, tragacanth, xanthan gum, methyl cellulose, hydroxymethylcellulose, hydroxyethylcellulose, hydroxypropylcellulose, carboxymethylcellulose, water soluble chitin, chitosan and dextrin.

The fat-soluble polymer may include polyvinylpyrrolidone-eicosene copolymer, polyvinylpyrrolidone-hexadecene copolymer, nitrocellulose, dextrin fatty acid ester, and silicone.

The emollients may include long chain acyl glutamic acid cholesteryl ester, cholesteryl hydroxystearate, 12-hydroxystearic acid, stearic acid, rosin acid, and lanolin fatty acid cholesteryl ester.

The surfactants may include nonionic surfactants, anionic surfactants, cationic surfactants, amphoteric surfactants.

The nonionic surfactants may include self-emulsifying glycerin monostearate, propylene glycol fatty acid ester, glycerin fatty acid ester, polyglycerol fatty acid ester, sorbitan fatty acid ester, polyoxyethylene(POE) sorbitan fatty acid ester, POE sorbit fatty acid ester, POE glycerin fatty acid ester, POE alkyl ether, POE fatty acid ester, POE hydrogenated castor oil, POE castor oil, polyoxyethylene-polyoxypropylene(POE-POP) copolymer, POE-POP alkyl ether, polyether-modified silicone, lauric acid alkanolamide, alkylamine oxide, and hydrogenated soybean phospholipids.

The anionic surfactants may include fatty acid soap, alpha acyl sulfonate, alkyl sulfonate, alkyl allyl sulfonate, alkyl naphthalene sulfonate, alkyl sulfate, POE alkyl ether sulfate, alkylamide sulfate, alkyl phosphate, POE alkyl phosphate, alkylamide phosphate, alkyloylalkyltaurine salt, N-acylamino acid salt, POE alkyl ether carboxylate, alkyl sulfosuccinate, sodium alkyl sulfoacetate, acylated hydrolyzed collagen peptide salt, and perfluoroalkyl phosphate ester.

The cationic surfactants may include alkyl trimethylammoniumchloride, stearyltrimethylammonium chloride, stearyltrimethylammonium bromide, cetostearyltrimethylammonium chloride, distearyldimethylammonium chloride, stearyldimethylbenzylammonium chloride, behenyltrimethylammonium bromide, benzalkonium chloride, diethylaminoethyl stearate, stearic acid dimethylaminopropylamide, and lanolin derivative quaternary ammonium salt.

The amphoteric surfactants may include carboxybetaine type, amidebetaine type, sulfobetaine type, hydroxysulfobetaine type, amidesulfobetaine type, phosphobetaine type, aminocarboxylate type, imidazoline derivative type, and amidamine type.

The organic and inorganic pigments may include: the inorganic pigments such as silicic acid, silicic anhydride, magnesium silicate, talc, sericite, mica, kaolin, bengala, clay, bentonite, titanium coated mica, bismuth oxychloride, zirconium oxide, magnesium oxide, zinc oxide, titanium oxide, aluminum oxide, calcium sulfate, barium sulfate, magnesium sulfate, calcium carbonate, magnesium carbonate, iron oxide, ultramarine blue, chromium oxide, chromium hydroxide, calamine and their compounds; and the organic pigments such as polyamide, polyester, polypropylene, polystyrene, polyurethane, vinyl resin, urea resin, phenolic resin, fluororesin, silicon resin, acrylic resin, melamine resin, epoxy resin, polycarbonate resin, styrene-divinylbenzene copolymer, silk powder, cellulose, CI pigment yellow, and CI pigment orange; and organic and inorganic composite pigments.

The organic powders may include: metal soap such as calcium stearate; alkyl phosphate metal salt such as sodium zinc cetylate, zinc laurylate and calcium laurylate; acylamino acid polyvalent metal salt such as N-lauroyl-beta-alanine calcium, N-lauroyl-beta-alanine zinc, and N-lauroyl glycine calcium; amidesulfonic acid polyvalent metal salt such as N-lauroyl-taurine calcium and N-palmitoyl-taurine calcium; N-acyl basic amino acid such as N-epsilon-lauroyl-L-lysine, N-epsilon-palmitoylizine, N-alpha-paritoylolnithine, N-alpha-lauroylarginine, and N-alpha-hydrogenated beef fatty acid acylarginine; N-acyl polypeptide such as N-lauroylglycylglycine; alpha-amino fatty acids such as alpha-aminocaprylic acid and alpha-aminolauric acid; and polyethylene, polypropylene, nylon, polymethyl methacrylate, polystyrene, styrene-divinylbenzene copolymer, and ethylene tetrafluoride.

The ultraviolet absorbers may include: para-aminobenzoic acid, ethyl para-aminobenzoate, amyl para-aminobenzoate, octyl para-aminobenzoate, ethylene glycol salicylate, phenyl salicylate, octyl salicylate, benzyl salicylate, butylphenyl salicylate, homomentyl salicylate, benzyl cinnamate, paramethoxycinnamic acid-2-ethoxyethyl, octyl para methoxycinnamate, dipara-methoxycinnamic acid mono-2-ethylhexaneglyceryl, isopropyl paramethoxycinnamate, diisopropyl cinnamic acid ester mixture, urocanic acid, ethyl urokanate, hydroxymethoxybenzophenone, hydroxymethoxybenzophenonesulfonic acid and their salt form; and dihydroxymethoxybenzophenone, sodium dihydroxymethoxybenzophenonedisulfonate, dihydroxybenzophenone, tetrahydroxybenzophenone, 4-tert-butyl-4′-methoxydibenzoylmethane, 2,4,6-trianilino-p-(carbo-2′-ethylhexyl-1′-oxy)-1,3,5-triazine, and 2-(2-hydroxy-5-methylphenyl)benzotriazole.

The fungicides may include hinokitiol, triclosan, trichlorohydroxydiphenyl ether, chlorhexidine gluconate, phenoxyethanol, resorcin, isopropylmethylphenol, azulene, salicylic acid, zinc phyllithione, benzalkonium chloride, photosensitizer No. 301, mononitroguarechol sodium, and undecyrenic acid.

The antioxidants may include butylhydroxyanisole, propyl gallic acid, and elisorbic acid.

The pH adjusters may include citric acid, sodium citrate, malic acid, sodium malate, fumarate, sodium fumarate, succinic acid, sodium succinate, sodium hydroxide, and sodium monohydrogen phosphate.

The alcohols may include a higher alcohol such as cetyl alcohol.

Blending ingredients are not limited to the ones mentioned above, and any above-mentioned ingredient may be used for blending within the scope of not damaging the purpose and effect of the present disclosure. However, it would be preferable that the ingredient be blended at a blending ratio of 0.01; to 5% by weight, more preferably 0.01% to 3% by weight relative to the total weight of the cosmetic composition.

The cosmetic composition of the embodiment may take the form of solutions, suspensions, or viscous mixtures.

The cosmetic composition of the embodiment includes the ingredients described above as active ingredients but may also include other ingredients that are commonly used for cosmetics. For example, adjuvants and carriers such as stabilizers, solubilizers, vitamins, pigments, and fragrances may be included.

When the formulation comes in the form of paste, cream, or gel, carriers may be used. Examples of the carrier include animal fiber, vegetable fiber, wax, paraffin, starch, tracanth, cellulose derivative, polyethylene glycol, silicone, bentonite, silica, talc, and zinc oxide.

When the formulation comes in the form of powder or spray, the carrier may be lactose, talc, silica, aluminum hydroxide, calcium silicate, or polyamide powder. Especially in the case of spray, propellants such as chlorofluorohydrocarbon, propane-butane or dimethyl ether may be added.

When the formulation comes in the form of solution or suspension, the carrier may be a solvent, a solvating agent, or an emulsifying agent. Namely, water, ethanol, isopropanol, ethyl carbonate, ethyl acetate, benzyl alcohol, benzyl benzoate, propylene glycol, 1,3-butyl glycol oil, glycerol aliphatic esters, polyethylene glycol, or sorbitan fatty acid esters may be used.

When the formulation comes in the form of suspension, liquid diluent such as water, ethanol, propylene glycol, suspending agent such as ethoxylated isostearyl alcohol, polyoxyethylene sorbitol ester, polyoxyethylene sorbitan ester, microcrystalline cellulose, aluminum metahydroxide, bentonite, agar, and tracanth may be used as carriers.

When the formulation comes in the form of surfactant-containing cleansing, fatty alcohol sulfate, fatty alcohol ether sulfate, sulfosuccinic acid monoester, isethionate, imidazolinium derivative, methyltaurate, sarcosinate, fatty acid amide ether sulfate, alkylamido betaine, fatty alcohol, fatty acid glyceride, fatty acid diethanolamide, vegetable oil, lanolin derivatives, and ethoxylated glycerol fatty acid esters may be used as carriers.

In addition, the present invention provides a pharmaceutical composition containing various compositions derived from Bifidobacterium longum ATG-F5 strain. The compositions derived from Bifidobacterium longum ATG-F5 strain may be added to the pharmaceutical composition of the present invention in an amount of 0.001% to 30% by weight.

The pharmaceutical composition may be a composition for preventing or treating a disease selected from the group consisting of dry skin, acne, and inflammatory diseases, and the inflammatory disease may be one that is selected from the group consisting of inflammatory bowel disease, inflammatory collagen vascular disease, glomerulonephritis, inflammatory skin disease, sarcoidosis, retinitis, gastritis, hepatitis, enteritis, arthritis, tonsillitis, pharyngitis, bronchitis, pneumonia, pancreatitis, sepsis, cystitis, nephritis and neuritis.

The pharmaceutical composition may be formulated and used in the form of oral dosage forms such as powders, granules, tablets, capsules, suspensions, suspensions, syrups, aerosols, etc., external preparations, suppositories, etc., according to a conventional method. Carriers, excipients and diluents that may be included in the pharmaceutical composition include lactose, dextrose, sucrose, sorbitol, mannitol, xylitol, erythritol, maltitol, starch, gum acacia, alginate, gelatin, calcium phosphate, calcium silicate, cellulose, methyl cellulose, microcrystalline cellulose, polyvinyl pyrrolidone, water, methylhydroxybenzoate, propylhydroxybenzoate, talc, magnesium stearate, and mineral oils. In the case of formulation, it is prepared using diluents or excipients such as fillers, extenders, binders, wetting agents, disintegrants, and surfactants that are usually used. Solid formulations for oral administration include tablets, pills, powders, granules, capsules, and the like, and these solid formulations are prepared by mixing at least one excipient, such as starch, calcium carbonate, sucrose, or lactose, gelatin or the like in the strain of the present invention. In addition to simple excipients, lubricants such as magnesium stearate and talc are also used. Liquid formulations for oral use include suspensions, liquid solutions, suspensions, syrups, etc. In addition to water and liquid paraffin, which are commonly used simple diluents, various excipients such as wetting agents, sweetening agents, fragrances, and preservatives may be included. Formulations for parenteral administration include sterile aqueous solutions, non-aqueous solutions, suspensions, suspensions, lyophilized preparations, suppositories, and vaginal suppositories. As the non-aqueous solvent and suspending agent, propylene glycol, polyethylene glycol, vegetable oil such as olive oil, and injectable ester such as ethyl oleate may be used. As a base for suppositories, Witepsol, macrogol, tween 61, cacao butter, laurin, glycerogelatin, and the like may be used.

The dosage of the pharmaceutical composition of the present invention will vary depending on the age, sex, and weight of the subject to be treated, the specific disease or pathology to be treated, the severity of the disease or pathology, the route of administration, and the judgment of the prescriber. Dosage determination based on these factors is within the level of a person skilled in the art, and dosages generally range from 0.01 mg/kg/day to approximately 2000 mg/kg/day. A more preferred dosage is from 1 mg/kg/day to 500 mg/kg/day. Administration may be administered once a day, or may be divided into several times. The above dosage does not in any way limit the scope of the present invention.

The pharmaceutical composition of the present invention may be administered to mammals such as mice, livestock, and humans by various routes. Since the strain of the present invention has little toxicity and side effects, it is a drug that can be safely used even when taken for a long time for prophylactic purposes.

In addition, the present invention provides a health functional food having an effect of strengthening skin barrier, relieving skin dryness, anti-inflammatory, anti-acne, antioxidant or wrinkle-improving effect, containing a composition derived from Bifidobacterium longum ATG-F5. The composition derived from the Bifidobacterium longum ATG-F5 may be added to the health functional food of the present invention in an amount of 0.001% to 50% by weight. The health functional food of the present invention includes forms such as tablets, capsules, pills or liquids, and foods to which the composition derived from the strain of the present invention can be added include, for example, meat, sausage, bread, candies, snacks, noodles, ice cream, dairy products, fermented milk, soups, ionized beverages, beverages, alcoholic beverages, gum, tea and vitamin complexes.

### Advantageous Effects

The present invention pertains to a novel Bifidobacterium longum ATG-F5 strain. The F5 strain is a functional beneficial bacterium which is safe from antibiotic resistance, improves skin health through antibacterial activity against the skin pathogen Cutibacterium acnes, an antioxidant effect on radicals, skin barrier improvement, and anti-inflammatory effects, and enhances skin beauty through a skin whitening function, a wrinkle improvement function, and a dry skin alleviation function. The strain can be used in a cosmetic composition or health functional food.

### DESCRIPTION OF DRAWINGS

FIG. 1 shows a 16S rRNA nucleotide sequence of a Bifidobacterium longum ATG-F5 strain of the present invention;

FIG. 2 shows the results of confirming the antibacterial properties of the Bifidobacterium longum ATG-F5 strain of the present invention against two species of acne-causing bacteria, Cutibacterium acnes;

FIG. 3 shows the results of confirming the antioxidant function of the Bifidobacterium longum ATG-F5 strain of the present invention, in which the antioxidant function is evaluated as ABTS scavenging activity;

FIG. 4 shows the results of confirming the effect of increasing melanin production (FIG. 4A) related to the whitening function of the Bifidobacterium longum ATG-F5 strain of the present invention and the inhibitory effect of the Bifidobacterium longum ATG-F5 strain on tyrosiana activity (FIG. 4B);

FIG. 5 shows the results of confirming the pro-collagen synthesis ability and the MMP-1 inhibitory efficacy associated with the wrinkle improvement function of the Bifidobacterium longum ATG-F5 strain of the present invention;

FIG. 6 shows the results of confirming the effect of increasing the gene expression of occludin (OCLN) and claudin 4 (CLDN4) associated with the skin barrier strengthening function of the Bifidobacterium longum ATG-F5 strain of the present invention;

FIG. 7 shows the results of confirming the effect that the Bifidobacterium longum ATG-F5 strain of the present invention increases the gene expression of Hyaluronan synthase 2 (HAS2) associated with the dry skin alleviation function; and

FIG. 8 shows the anti-inflammatory effect (Interleukin 10/IL-10 increase) of the Bifidobacterium longum ATG-F5 strain of the present invention.

### BEST MODE

Hereinafter, preferred embodiments of the present invention will be described in detail. However, the present invention is not limited to the embodiments described herein and may be embodied in other forms. The embodiments are provided so that the present disclosure will be thorough and complete and will fully convey the spirit of the invention to those skilled in the art.

### Example 1. Culture of Microorganisms for Functional Evaluation

Bifidobacterium longum (hereinafter, referred to as F5) was isolated from donated neonatal feces (of babies born in 2018, Daejeon, Korea). To this end, neonatal feces was diluted with 0.9% (w/v) saline by 10-fold serial dilution method, the resulting solution was smeared on a Bifidobacterium selective (BS, MBcell Seoul, Korea) solid medium, and bacteria were incubated at 37° C. for 48 hours. The colonies of bacteria generated in the BS medium were observed with a microscope. Through the observation, bacteria not exhibiting a catalase reaction and bacillus-type bacteria were selected and named ATG-F5 strain (hereinafter simply referred to as F5). For experiment, the F5 strain was first cultured in a BL or MRS-cys agar solid medium, followed by inoculation of purely isolated colony in a broth liquid medium. The microorganisms were cultured at 37° C. overnight (i.e., for 16 to 20 hours).

### Example 2. Confirmation of Characteristics of F5 Strain

### Example 2-1. 16S rRNA Sequencing of F5 Strain

The 16S rRNA sequence analysis of the F5 strain was performed by Solgent Co., Ltd. (located in Dajeon, Korea) at request. Primers for sequencing analysis were 27F (5′-AGA GTT TGA TCC TGGCTC AG-3′), 518F (5′-CCA GCA GCC GCG GTA ATA C-3′), 907R (5′-CCGTCA ATT CMT TTR AGT TT-3′), 1492R (5′-GGT TAC CTT GTT ACG ACT T-3′), and the nucleotide sequences were read for a total of 4 times. The contig nucleotide sequence derived through the nucleotide sequence alignment of each reading was analyzed using the BLAST online tool (https://blast.ncbi.nlm.nih.gov/Blast.cgi) of National Center For Biotechnology Information (NCBI).

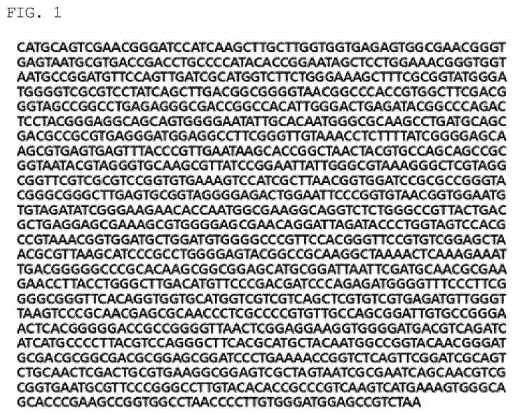

The nucleotide sequence of SEQ ID NO: 1 (see FIG. 1) obtained through the 16S rRNA sequencing was compared against the BLAST database of NCBI. As a result, the Bifidobacterium longum strain IRT and the 16S rRNA sequence exhibited 99.9% matching, thereby indicating that the tested sequence belongs to Bifidobacterium longum when classified by the molecular taxonomy.

Accordingly, on Mar. 18, 2019, the strain of the present invention was deposited with the Korea Institute of Biological Resources under accession number: KCTC13828BP.

### Example 2-3. Sugar Fermentation Pattern of F5 Strain

In addition, a slightly modified API50 CHL test (BioMerieux, France) was performed to determine the sugar fermentation pattern. Briefly, L-cystein was added to a 10 mL API 50CHL medium (BioMerieux, France) at a concentration of 0.5 μg/ml, and the pH was adjusted to about 6.7. Next, the purely cultured F5 strain was suspended to have an OD₆₀₀ absorbance of about 0.5, and the suspension culture solution was inoculated into each cupule of the API 50CH test strip and cultured at 37° C. The sugar fermentation results were checked when 48 hours and 72 hours passed after the inoculation.

As shown in Table 1, the result of the evaluation of sugar fermentation using an API kit reveals that the F5 strain degrades L-arabinose, ribose, galactose, glucose, fructose, mannose, mannitol, sorbitol, methyl-αD-mannopyranoside, N-acetylglucosamine, amygdalin, arbutin, esculin, salicin, cellobiose, maltose, lactose, melibiose, trehalose, melezitose, raffinose, and turanose. The F5 strain also weakly degrades D-arabinose, methyl-αD-glucopyranoside, gentibiose, L-fucose, and gluconate.

TABLE 1

| Carbohydrates | Bifidobacterium longam ATG-F5 |
|---|---|
| Glycerol | − |
| Erythritol | − |
| D-Arabinose | w |
| L-Arabinose | + |
| Ribose | + |
| D-Xylose | − |
| L-Xylose | − |
| Adonitol | − |
| Methyl-βD-Xylopyranoside | − |
| Galactose | + |
| Glucose | + |
| Fructose | + |
| Mannose | + |
| Sorbose | − |
| Rhamnose | − |
| Dulcitol | − |
| Inositol | − |
| Mannitol | + |
| Sorbitol | + |
| Methyl-αD-Mannopyranoside | + |
| Methyl-αD-Glucopyranoside | w |
| N-Acetylglucosamine | + |
| Amygdalin | + |
| Arbutin | + |
| Esculin | + |
| Salicin | + |
| Cellobiose | + |
| Maltose | + |
| Lactose | + |
| Melibiose | + |
| Sucrose | + |
| Trehalose | + |
| Inulin | − |
| Melezitose | + |
| Raffinose | + |
| Starch | − |
| Glycogen | − |
| Xylitol | − |
| Gentiobiose | w |
| Turanose | + |
| Lyxose | − |
| Tagatose | − |
| D-Fucose | − |
| L-Fucose | w |
| D-Arabitol | − |
| L-Arabitol | − |
| Gluconate | w |
| 2-keto-glugonate | − |
| 5-keto-gluconate | − |

* Sugar fermentation pattern of Bifidobacterium longum ATG-F5 (Positive: +, Weakly positive: w, Negative: −)

### Example 3. Evaluation of Antibacterial Activity of F5 Strain

In order to check the antibacterial activity of the F5 strain among various functional evaluation items associated with skin irritation, the antibacterial activity of the F5 strain against a total of two types of infectious or opportunistic bacteria, specifically, acne causative bacteria (Cutibacterium acnes, KCTC5012 and KCTC3314) was evaluated through a disc test. Through the disc test, a clear zone was identified. Two types of bacteria cultured overnight in BL broth (MBcell Seoul, Korea) plate media were each suspended in 1× phosphate buffered saline (PBS) at an OD₆₀₀ absorbance of about 0.8. Each suspension was absorbed with a sterile cotton swab, was spread and dried over on an agar medium in which BL and MRS were mixed in a ratio of 1:1 for an antibacterial activity test, and an 8 mm paper disc (Advantec, Japan) was attached to the dried agar medium for testing. The F5 bacterial solutions cultured in the BL broth for 18 to 20 hours was inoculated on paper discs, by 35 μl per disc, were dried for about 3 minutes, and incubated at 37° C. The diameter of the clear zone generated after the incubation was measured, and 8 mm was subtracted from the measured clear zone diameter to obtain a final value.

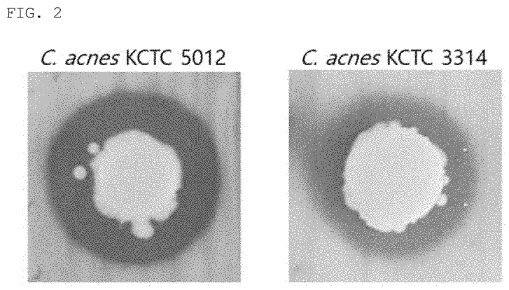

Regarding antibacterial activity, the antibacterial activity of the F5 strain was evaluated using Cutibacterium acnes (KCTC 5012 and 3314) strains. The F5 strain exhibited an antibacterial activity of a 9 to 10 mm clear zone and an antibacterial activity of a 5 to 6 mm clear zone with respect to KCTC5012 and KCTC3314, respectively (see Table 2 and FIG. 2).

TABLE 2

| | Clear zone against | Clear zone against |
|---|---|---|
| Experiment | Cutibacterium acnes | Cutibacterium acnes |
| round | “KCTC 5012” | “KCTC 3314” |
| First round | 9 mm | 6 mm |
| Second | 9 mm | 5 mm |
| round | | |
| Third round | 10 mm | 6 mm |
| Average | 9.3 mm | 5.7 mm |

### Example 4. Antibiotic Resistance Safety of F5 Strain

An antibiotic test was performed using an E-test strip (BioMerieux, France) or an MIC test strip (Liofilchem, Italy) of 9 antibiotics including ampicillin, vancomycin, gentamicin, kanamycin, streptomycin, clindamycin, erythromycin, tetracycline, and chloramphenicol, and the minimum inhibitory concentration (MIC) values were obtained.

For this, the F5 strain was suspended to be at an OD₆₀₀ absorbance of about 0.8 and smeared on a BL solid medium using a sterilized cotton swab. The solid medium on which the F5 strain was smeared was dried for about 3 minutes, and the E-test strip or MIC test strip was mounted and incubated at 37° C. for 24 to 48 hours.

The guidelines published by the European food safety authority (EFSA) were referenced for the types of antibiotics and the minimum inhibitory concentration that can be considered safe (EFSA Panel on Additives and Products or Substances used in Animal Feed, 2012).

As shown in Table 3, the results of measuring the susceptibility of the 9 antibiotics were found to be consistent with the standard values of the guidelines presented by the EFSA. The unit of each numerical value in Table 3 is μg/ml.

TABLE 3

| Strains | AMP | VAN | GEN | KAN | STR | CD | ERY | TET | CM |
|---|---|---|---|---|---|---|---|---|---|
| Bifidobacterium | 0.75 | 0.75 | 8 | NR | 8 | 0.016 | 0.023 | 0.5 | 0.38 |
| longum ATG-F5 | | | | | | | | | |
| EFSA standard | 2 | 2 | 64 | NR | 128 | 1 | 1 | 8 | 4 |

AMP, ampicillin;
VAN, vancomycin;
GEN, gentamicin;
KAN, kanamycin;
STR, streptomycin;
CD, clindamycin;
ERY, erythromycin;
TET, tetracycline;
CM, chloramphenicol;
NR, not required.

### Example 5. Preparation of Cell Lysate of F5 Strain

To evaluate each function, a 10-fold concentrated cell free supernatant (CFCS) was prepared.

To this end, the culture medium of the F5 strain was centrifuged at 4,000 rpm for 25 minutes so that the culture medium is separated into the F5 strain and the culture supernatant. The pH of the supernatant was adjusted to 7 to 8 and filtered using a 0.2 μm-pore syringe filter (Satorious, Germany). The filtered solution was stored at −20° C. The cells were suspended in 1×PBS, washed with vortex, centrifuged at 4° C. and 4,000 rpm for 5 minutes to remove the remaining culture medium, and suspended again in 1×PBS. Lysozyme (Sigma-Aldrich, Germany) was dispensed to the suspended cells at a concentration of 500 μg/ml, and the resulting suspension was incubated for 2 hours in an incubator at 37° C., and then was introduced into a sonicator (70 W, 30 seconds, and 30 cycles) to undergo ultrasonic cytolysis. As a result, a lysate was prepared. Next, the lysate was heat-treated at 60° C. for 45 minutes to kill bacteria that may still remain alive in the lysate. The 60° C. heat treatment was performed in a water bath to minimize protein denaturation.

Thereafter, the cell lysate was stored at −20° C. and thawed for the experiment. The cell lysate was used for all subsequent experiments. In addition, the moisture content of the suspension was measured to dispense the suspension at different concentrations relative to the solid content during use, and the solid content was calculated for dilution.

### Example 6. Confirmation of Antioxidant Function of F5 Strain

A radical scavenging experiment using ABTS ([2, 2′-Azino-Bis (3-Ethylbenzthiazoline-6-sulfonic acid], Sigma-Aldrich, Germany) was planned to measure the antioxidant function of the F5 strain. This is an experiment using the principle of discoloration from turquoise to colorless when the cationic radicals of the ABTS are removed by reacting with antioxidant materials (refer to Nam et al., 2015).

In this experiment, the antioxidant ability of the lysate of the F5 strain was measured using the ABTS solution. To prepare for the ABTS experiment, a mixed solution was prepared by mixing 14 mM ABTS stock solution and 4.9 mM potassium persulfate in a 1:1 ratio, and a dark reaction was carried out overnight so that the mixed solution turns into blue-green. After that, a working solution was prepared by diluting the solution to exhibit an absorbance of about 0.7 at an OD 734 wavelength. Next, 10% (v/v) of the lysate sample of the F5 strain was added to the working solution, and reacted in the dark for 10 minutes. Next, the absorbance of the resulting solution was measured at a wavelength of 734 nm, and each measurement value was calculated by the following formula.

ABTS radical scavenging activity (%)={1−(OD sample/OD control)}×100

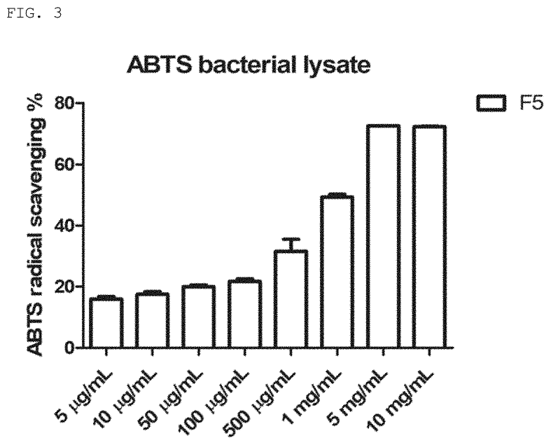

Using this method, the antioxidant activity of the Bifidobacterium longum ATG-F5 lysate measured by the ABTS radical removal rate is shown in FIG. 3.

Referring to FIG. 3, it can be seen that the radical scavenging activity pattern starts to increase significantly from the concentration of 500 μg/ml of the lysate of the strain, and the antioxidant function is significantly enhanced from 1 mg/ml.

### Example 7. Evaluation of Melanin Inhibition and Whitening Function Using Tyrosinase Inhibitor of F5 Strain

To evaluate whitening function, B16F10 Melanoma cells, which is a cell line of a mouse model, was used. B16F10 is a representative cell line for evaluation of the whitening function. The whitening function is evaluated using a mechanism in which in the cell signaling pathway, L-Tyrosine starts from the tyrosinase activation pathway and increases melanin production at the last reaction point.

For this evaluation, B16F10 was cultured in a 75 cm 2 flask (Thermo fisher, USA) and in a culture medium prepared by adding 10% (v/v) fetal bovine serum (Sigma-Aldrich, Germany) and 1% (v/v) penicillin/streptomycin cocktail (Sigma-Aldrich, Germany) to Gibco® Dulbecco modified eagle medium (DMEM, Gibco, USA). Then, when the cells grew about 80%, the cells were harvested and subcultured. The cells were seeded in a 6-well plate at a concentration of 2×10⁶ cells/ml, and then incubated in an incubator under 5% CO₂ conditions for 24 hours.

After that, the culture medium was removed and the plate was washed using Dulbecco's Phosphate Buffered Saline (DPBS, Gibco, USA). Then, a control group (untreated) and a test group treated with α-melanocyte stimulating hormone (α-MSH, Sigma-Aldrich, USA) were prepared. The α-MSH increases the production of melanin, thereby causing skin discoloration. To the test group, 200 nmol/ml of α-MSH and 50, 100, and 500 μg/ml of the cell lysate were dispensed at each concentration. The treatment time of the α-MSH and the cell lysate of the strain was 24 hours, and the cells were incubated at 37° C. in an incubator in an 5% C₂ environment.

After 24 hours, the supernatant in each well was removed, and washing was performed using DPBS. 500 μl of 0.1M PBS added with 1% (v/v) triton X-100 (Daejung, Korea) was dispensed into the washed wells to obtain cells using a microtube. The cells were centrifuged, the supernatant was separately stored, and the pellets were collected. Next, using the collected cell pellets, melanin production was evaluated. 1N NaOH was added to pure pellets. The cell pellets were melted in an 80° C. water bath for about 1 hour, then put into ice to cool, and was subjected to absorbance measurement at a wavelength of 405 nm. The analysis was performed through relative comparison.

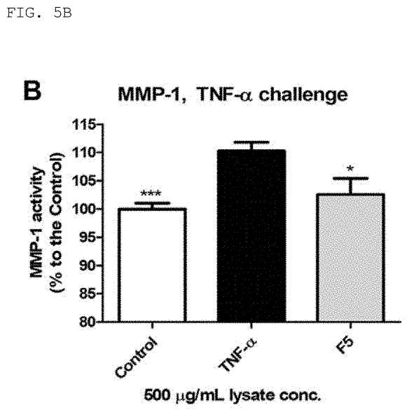

As a result, when melanin production was induced by stimulating tyrosinase activity with α-MSH using Mouse B16F10 melanoma cells, melanin production was reduced according to the treatment concentration of the cell lysate of the F5 strain as shown in FIG. 5A. In particular, even though melanin production was stimulated with α-MSH during the treatment of each strain, the production of melanin was further inhibited, indicating that there was a significant whitening effect.

After collecting the cell pellets, the supernatant collected separately was used for tyrosinase inhibitor measurement. 40 μl of the supernatant and 160 μl of dihydroxyphenylalanine (L-DOPA, Sigma, USA) dissolved at 2 μg/ml in 0.1M PBS were mixed and reacted for 1 hour in an incubator at 37° C. After that, an ELISA reader (BioTek, USA) was used to measure the absorbance at 475 nm to show relative the inhibitory ability of the tyrosinase inhibitor.

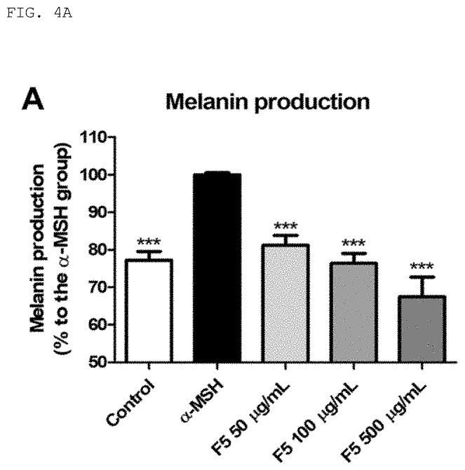

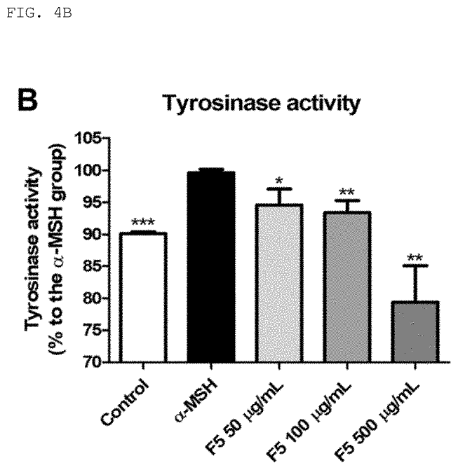

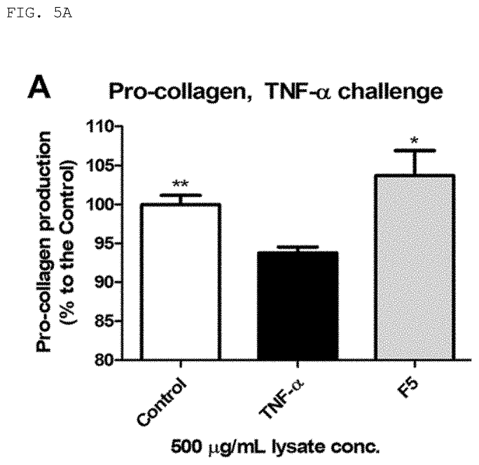

Accordingly, as shown in the result of FIG. 4B, it is confirmed that tyrosinase activity inhibition is obtained after treatment of the supernatant, which can prove that melanin production is inhibited because tyrosinase activity is inhibited.

### Example 8. Evaluation of Pro-Collagen Increase and Wrinkle Improvement Function Through Inhibition of MMP-1 by F5 Strain

To evaluate a wrinkle improvement function, a human model cell line, Human Dermal Fibroblast (HDF) cell line, was used. A Gibco® Dulbecco modified eagle medium (DMEM, Gibco, Germany) supplemented with 10% (v/v) fetal bovine serum (Sigma-Aldrich, Germany) and 1% (v/v) penicillin/streptomycin cocktail (Sigma-Aldrich, Germany) USA) was used to culture the cells. In addition, for the culture of the cells, a 75 cm 2 Flask (Thermo fisher, USA) was used. When the cells grew about 80% in the 75 cm 2 flask, the cells were harvested and subcultured.

Cells for the experiment were seeded in a 24-well plate at a concentration of 2×10⁵ cells/ml, and then cultured using an incubator under 5% CO₂ conditions for 24 hours. After that, the culture medium was removed and the plate was washed using Dulbecco's Phosphate Buffered Saline (DPBS, Gibco, USA). A control group (untreated group) and a test group treated with 10 ng/ml of TNF-α (Human Tumor necrosis factor alpha, Sigma-Aldrich, USA) were prepared. The TNF-α increases MMP-1 to create skin wrinkles.

For the test group, 50, 100, and 500 μg/ml of the cell lysate of the F5 strain along with 10 ng/ml of TNF-α were dispensed at each concentration. The lysate of the TNF-α and the F5 strain were treated for 24 hours, and incubated at 37° C. in a 5% CO₂ condition using an incubator.

After 24 hours, the supernatant in each well was collected, and enzyme-linked immunosorbent assay (ELISA) for matrix metalloproteinase-1 (MMP-1) and Type 1 collagen was performed. MMP-1 was relatively quantified using Human Pro-MMP-1 Quantikine ELISA Kit (R&D systems, USA), and Type 1 collagen was relatively quantified using Human Procollagen Type I C-peptide (PIP) EIA Kit (Takara, JAPAN).

As a result, as shown in FIG. 5A, when the cell lysate of the F5 strain was treated with TNF-α at a concentration of 500 μg/ml, it was confirmed that the pro-collagen was increased. The pro-collagen was increased to the level that can be exhibited by the control (untreated group). That is, the increase was considerable compared to the treatment with the TNF-α alone.

In particular, as shown in FIG. 5B, in the test group treated with the cell lysate of the F5 strain together with TNF-α, the expression of MMP-1 was restored to the level of the control group (untreated group) compared to the TNF-α alone treatment group. This proves that even though the increase in MMP-1 was induced through the TNF-α challenge, the cell lysate of the F5 strain affects the restoration (or reduction) of the pro-collagen to an amount corresponding to the control, and also proves that the lysate of the F5 strain has the effect of greatly enhancing the production ability of Type 1 collagen.

### Example 9. Evaluation of Skin Barrier Strengthening or Skin Dryness Relief Function of F5 Strain

Human keratinocyte, HaCaT cell line, was used for skin function evaluation. The cells were cultured in the same way as described above. When the cells grew about 80% in a 75 cm 2 flask, the cells were harvested and subcultured. The cells were seeded on a 10 cm round plate at a concentration of ×10⁶ cells/ml, and then cultured using an incubator in a 5% CO₂ condition for 24 hours. Thereafter, the culture medium was removed and the plate was washed with Dulbecco's Phosphate Buffered Saline (DPBS, Gibco, USA). The cell lysate of the F5 strain was dispensed at concentrations of 50, 100, and 500 μg/ml. The control group was not treated at all. The lysate of the F5 strain was treated for 24 hours, and cultured using an incubator at 37° C. in a 5% CO₂ condition.

Next, the plate was washed, and the total RNA of the cells was isolated using TRIzol® Reagent (Ambion, USA). After the isolated RNA was quantified to synthesize cDNA, it was synthesized into cDNA using SuperScript™ IV First-Strand Synthesis System (Invitrogen, USA).

After that, to identify skin-related functional genes, quantitative real-time PCR (qRT-PCR) was performed using oligo primers for occludin (OCLN) and claudin 4 (CLDN4) related to skin barrier, and Hyaluronan synthase 2 (HAS2) related to skin dryness. For the PCR analysis, a fast real-time PCR system, Applied Biosystems 7500, was used. In this case, β-actin was used as a housekeeping gene. The list of primers used in the experiment is shown in Table 4.

TABLE 4
Base sequence of primers
used to measure gene expression

| | Sense | Anti-sense | |
|---|---|---|---|
| Gene | (5′→3′) | (5′→3′) | Size (bp) |
| β-actin | TCTACGAGGGG | GGATGCCACAG | 330 |
| | TATGCCCTCC | GACTCCATGC | |
| OCLN | GACTTCAGGCA | GCCAGTTGTGT | 132 |
| | GCCTCGTTAC | AGTCTGTCTCA | |
| CLDN4 | TGGGGCTACAG | GGTCTGCGAGG | 145 |
| | GTAATGGG | TGACAATGTT | |
| HAS2 | CTCTTTTGGAC | AGGGTAGGTTA | |
| | TGTATGGTGCC | GCCTTTTCACA | 205 |

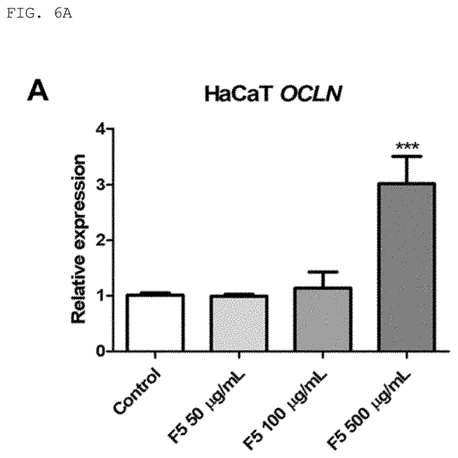

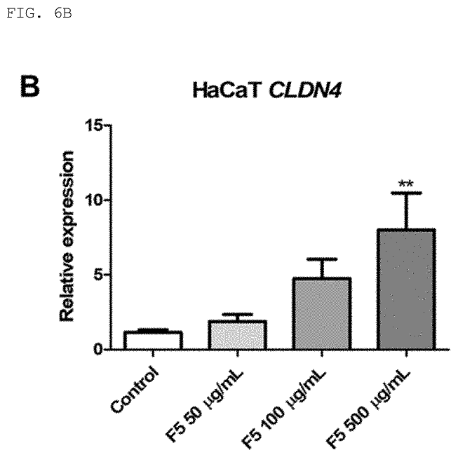

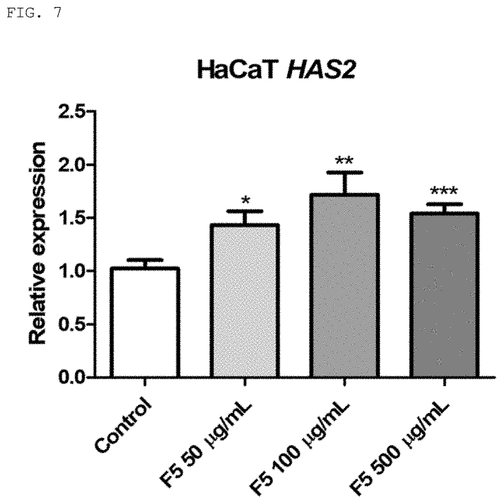

As a result, as shown in FIGS. 6 and 7, when HaCaT cells were treated with the lysate of the F5 strain at concentrations of 50, 100, and 500 μg/ml, OCLN (occludin), it is confirmed to increase the expression of CLDN4 (claudin 4) and Hyaluronan synthase 2 (HAS2) at a concentration of 500 μg/ml compared to the control (untreated group).

Therefore, the lysate of the F5 strain of the present invention has a function of remarkably strengthening the skin barrier by engaging in contact between skin cells due to the increase in the expression of OCLN and CLDN4. Similarly, it can be confirmed that the cell lysate of the F5 strain stimulates the expression of Hyaluronan synthase 2 (HAS2) to increase the production of Hyaluronic acid (HA) in the skin, thereby alleviating skin dryness.

### Example 10. Evaluation of Anti-Inflammatory Function of F5 Strain

For the evaluation of the anti-inflammatory function, the expression level of IL-10 was checked. The RAW264.7 cell line, which is a murine macrophage, was used. As the culture method, the HDF cell culture method described above was used. The cells were harvested and subcultured when they grew to about 80% in a 75 cm 2 flask. The cells were seeded in a 24-well plate at a concentration of 1×10⁶ cells/ml, and then were incubated in an incubator in a 5% CO₂ condition for 24 hours. First, as a control group, the untreated cells were prepared. As a positive control group, the cells with treated with only LPS (Lipopolysaccharide, Sigma-Aldrich, USA) was prepared. In addition, a group treated with the cell lysate of the F5 strain alone and a group treated with 100 μg/ml of the cell lysate of the F5 strain and 1 μg/ml of LPS were also prepared. After 24 hours, the supernatant in each well was collected and relatively quantified using Interleukin 10 (IL-10, R&D, USA).

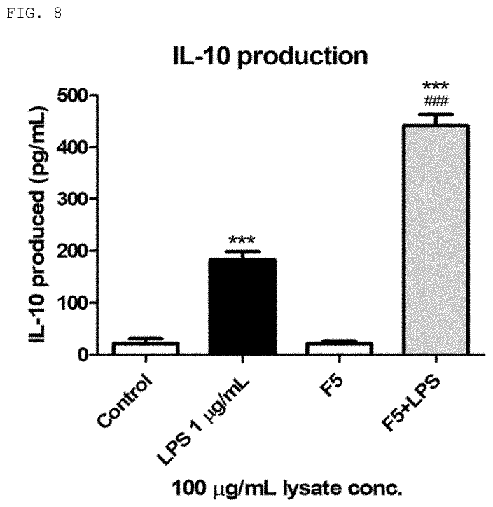

The results are shown in FIG. 8. In the case of the RAW264.7 cells for which the inflammatory response was induced by LPS, the production of IL-10, which is an anti-inflammatory cytokine, was more considerably increased when the cells were treated with both of the lysates of LPS and F5 strain than when the cells were treated with only LPS.

### Cosmetic Formulation Example 1. Preparation of Skinner Toner −1

Using the composition of Table 5 below, a skin toner (100 g) containing a lyophilized product of the cell lysate of the Bifidobacterium longum ATG-F5 strain was prepared according to a conventional method.

TABLE 5

| Raw material | Content (g) |
|---|---|
| Lyophilized product of cell lysate of | 3.0 |
| Bifidobacterium longum ATG-F5 strain | |
| Butylene glycol | 2.0 |
| Propylene glycol | 2.0 |
| Polyoxyethylene (60) hydrogenated castor oil | 1.0 |
| Ethanol | 10.0 |
| Triethanolamine | 0.1 |
| Antiseptic | Trace |
| Pigment | Trace |
| Perfume | Trace |
| Purified water | Remainder |

### Cosmetic Formulation Example 2. Preparation of Moisture Lotion

After extracting the lyophilized product of the cell lysate of the Bifidobacterium longum ATG-F5 strain with 70% (v/v) aqueous ethanol solution, a solvent-free concentrate was obtained, and a moisture lotion (100 g) containing the concentrate as shown in Table 6 was prepared according to a conventional method.

TABLE 6

| Raw material | Content (g) |
|---|---|
| Lyophilized product of cell lysate of | 1.0 |
| Bifidobacterium longum ATG-F5 strain | |
| Sitosterol | 1.7 |
| Polyglyceryl 2-oleate | 1.5 |
| Ceteares | 1.2 |
| Cholesterol | 1.5 |
| dicetyl phosphate | 0.4 |
| Concentrated glycerin | 5.0 |
| Sunflower Oil | 10.0 |
| Carboxyvinyl Polymer | 0.2 |
| Xanthan Gum | 0.3 |
| Antiseptic | Trace |
| Perfume | Trace |
| Purified water | Remainder |

### Cosmetic Formulation Example 3. Preparation of Nutrient Cream

Using the composition of Table 7 below, a nutrient cream (100 g) containing a lyophilized product of the cell lysate of the Bifidobacterium longum ATG-F5 strain was prepared according to a conventional method.

TABLE 7

| Raw material | Content (g) |
|---|---|
| Lyophilized product of cell lysate of | 5.0 |
| Bifidobacterium longum ATG-F5 strain | |
| Sitosterol | 4.0 |
| Polyglyceryl 2-oleate | 3.0 |
| Ceramide | 0.7 |
| Ceteares-4 | 2.0 |
| Cholesterol | 3.0 |
| Dicetyl phosphate | 0.4 |
| Concentrated glycerin | 5.0 |
| Sunflower Oil | 22.0 |
| Carboxyvinyl Polymer | 0.5 |
| Triethanolamine | 0.5 |
| Antiseptic | Trace |
| Perfume | Trace |
| Purified water | Remainder |

### Cosmetic Formulation Example 4. Preparation of Essence

Using the composition of Table 8 below, an essence (100 g) containing a lyophilized product of the cell lysate of the Bifidobacterium longum ATG-F5 strain was prepared according to a conventional method.

TABLE 8

| | Content |
|---|---|
| Raw material | (g) |
| Lyophilized product of cell lysate of | 1.0 |
| Bifdobacterium longum ATG-F5 strain | |
| Sitosterol | 1.7 |
| Polyglyceryl 2-oleate | 1.5 |
| Ceteares-4 | 2.0 |
| Cholesterol | 3.0 |
| Dicetyl phosphate | 0.4 |
| Concentrated glycerin | 5.0 |
| Sunflower Oil | 22.0 |
| Carboxyvinyl Polymer | 0.5 |
| Triethanolamine | 0.5 |
| Antiseptic | Trace |
| Perfume | Trace |
| Purified water | Remainder |

### Cosmetic Formulation Example 5. Preparation of Foundation

Using the composition of Table 9 below, a foundation (100 g) containing a lyophilized product of the cell lysate of the Bifidobacterium longum ATG-F5 strain was prepared according to a conventional method.

TABLE 9

| | Content |
|---|---|
| Raw material | (g) |
| Lyophilized product of cell lysate of | 1.0 |
| Bifidobacterium longum ATG-F5 strain | |
| Beeswax | 2.0 |
| Cyclomethicone | 2.0 |
| Liquid paraffin | 5.0 |
| Squalane | 5.0 |
| Stearic acid | 2.0 |
| Lipophilic monostearate glycerin | 3.0 |
| Caprylic/Capric Triglycerides | 4.0 |
| Glycerin | 4.0 |
| Propylene glycol | 3.0 |
| Butylene glycol | 3.0 |
| Triethanolamine | 1.0 |
| Aluminum Magnesium Silicate | 0.5 |
| Pigment | 12.0 |
| Antiseptic | Trace |
| Perfume | Trace |
| Purified water | Remainder |

### Cosmetic Formulation Example 6. Preparation of Hair Shampoo

Using the composition of Table 10 below, a hair shampoo (100 g) containing a lyophilized product of the cell lysate of the Bifidobacterium longum ATG-F5 strain was prepared according to a conventional method.

TABLE 10

| | Content |
|---|---|
| Raw material | (g) |
| Lyophilized product of cell lysate of | 3.0 |
| Bifidobacterium longum ATG-F5 strain | |
| Arachidyl Glucoside | 4.5 |
| Ethanol | 2.0 |
| Butylene glycol | 2.0 |
| Citric acid | 0.1 |
| Phenoxyethanol | 0.02 |
| Purified water | Remainder |

### Formulation Example 1. Pharmaceutical Formulation

### Formulation Example 1-1. Preparation of Tablets

200 g of a lyophilized product of the cell lysate of the Bifidobacterium longum ATG-F5 strain of the present invention was mixed with 175.9 g of lactose, 180 g of potato starch, and 32 g of colloidal silicic acid. After adding a 10% (w/v) gelatin solution to this mixture, the resulting product was ground and passed through a 14-mesh sieve. The remaining solid was dried, and 160 g of potato starch, 50 g of talc, and 5 g of magnesium stearate were added thereto. The resulting mixture was prepared as an anti-inflammatory tablet.

### Formulation Example 1-2. Preparation of Ointments

1 g of a lyophilized product of the cell lysate of the Bifidobacterium longum ATG-F5 strain of the present invention was mixed with 99 g of petrolatum to prepare an anti-inflammatory ointment.

### Formulation Example 2. Preparation of Food Product

### Formulation Example 2-1. Preparation of Cooking Seasonings

A health-functional cooking seasoning was prepared by adding a lyophilized powder of the cell lysate of the Bifidobacterium longum strain of the present invention to a cooking seasoning in an amount of 1% by weight.

### Formulation Example 2-2. Preparation of Dairy Products

A lyophilized powder of the cell lysate of the Bifidobacterium longum strain of the present invention was added to milk in an amount of 0.1% by weight, and various dairy products such as butter and ice cream were prepared using the milk.

### Formulation Example 2-5. Preparation of Vegetable Juice

A health-functional vegetable juice was prepared by adding 0.5 g of a lyophilized powder of the cell lysate of the Bifidobacterium longum strain of the present invention to 1,000 ml of tomato juice or carrot juice.

### Formulation Example 2-6. Preparation of Fruit Juice

Health-functional fruit juice was prepared by adding 0.1 g of a lyophilized powder of the cell lysate of the Bifidobacterium longum strain of the present invention to 1,000 ml of apple juice or grape juice.

[Depository Institution]

Name of Institution: The Korean Collection for Type Cultures

Accession Number: KCTC13828BP

Date of Deposit: Mar. 18, 2019

### [Amendment by Rule 91 28 Jun. 2019]

1. A Bifidobacterium longum ATG-F5 strain deposited under accession no. KCTC13828BP.

2. The Bifidobacterium longum ATG-F5 strain of claim 1, wherein the strain has a skin barrier strengthening function or a dry skin alleviation function.

3. The Bifidobacterium longum ATG-F5 strain of claim 1, wherein the strain has an anti-acne function.

4. The Bifidobacterium longum ATG-F5 strain of claim 1, wherein the strain has an antioxidant function.

5. The Bifidobacterium longum ATG-F5 strain of claim 1, wherein the strain has a skin whitening function.

6. The Bifidobacterium longum ATG-F5 strain of claim 1, wherein the strain has a wrinkle improvement function.

7. The Bifidobacterium longum ATG-F5 strain of claim 1, wherein the strain has anti-inflammatory function.

8. A cosmetic composition comprising the Bifidobacterium longum ATG-F5 strain of claim 1 which has functions comprising skin barrier strengthening, dry skin alleviation, anti-inflammatory, anti-acne, antioxidant, and wrinkle improvement.

9. The cosmetic composition of claim 8, wherein a formulation of the cosmetic composition is selected from the group consisting of skin lotion, skin softener, skin toner, astringent, milk lotion, moisture lotion, nourishing lotion, massage cream, nourishing cream, moisture cream, hand cream, foot cream, neck cream, foundation, essence, pack, soap, cleansing foam, cleansing lotion, cleansing cream, body lotion, hair shampoo, hair treatment, hair conditioner, and body cleanser.

10. A health functional food comprising the Bifidobacterium longum ATG-F5 strain of claim 1 which has functions comprising skin barrier strengthening, dry skin alleviation, anti-inflammatory, anti-acne, antioxidant, and wrinkle improvement.

11. The health functional food of claim 10, wherein the health functional food is selected from the group consisting of meat, sausage, bread, candies, snacks, noodles, ice cream, dairy products, fermented milk, soup, ionized beverage, beverage, alcoholic beverage, gum, tea, and vitamin complex.

12. A composition for preventing or treating a disease selected from the group consisting of dry skin, acne, and an inflammatory disease comprising the Bifidobacterium longum ATG-F5 strain of claim 1.

13. The composition of claim 12, wherein the inflammatory disease is selected from the group consisting of inflammatory bowel disease, inflammatory collagen vascular disease, glomerulonephritis, inflammatory skin diseases, sarcoidosis, retinitis, gastritis, hepatitis, enteritis, arthritis, tonsillitis, sore throat, bronchitis, pneumonia, pancreatitis, blood poisoning, cystitis, nephritis, and neuritis.

14. A method of preventing or treating a disease selected from the group consisting of dry skin, acne, and an inflammatory disease by administering a composition comprising the Bifidobacterium longum ATG-F5 strain of claim 1 to a subject in need thereof.

15. The method of claim 14, wherein the inflammatory disease is selected from the group consisting of inflammatory bowel disease, inflammatory collagen vascular disease, glomerulonephritis, inflammatory skin diseases, sarcoidosis, retinitis, gastritis, hepatitis, enteritis, arthritis, tonsillitis, sore throat, bronchitis, pneumonia, pancreatitis, blood poisoning, cystitis, nephritis, and neuritis.
